(19) United States
(12) Patent Application Publication
(10) Pub. No.: US 2009/0081646 A1
(43) Pub. Date: Mar. 26, 2009

(54) Universal primers and their use for detecting and identifying plant materials in complex mixtures

(75) Inventors: Pierre Taberlet, La Terrasse (FR); Ludovic Gielly, Eybens (FR); Christian Philippe Miquel, Chambery (FR)

Correspondence Address: OLIFF & BERRIDGE, PLC, P.O. BOX 320850, ALEXANDRIA, VA, 22320-4850, US

(73) Assignees: UNIVERSITE JOSEPH FOURIER, GRENOBLE (FR); CENTRE NATIONAL DE LA RECHERCHE SCIENTFIQUE, PARIS (FR)

(21) Appl. No.: 11/663,059

(22) PCT Filed: Oct. 6, 2005
(86) PCT No.: PCT/FR2005/002470

§ 371 Date: Apr. 6, 2007

(30) Foreign Application Priority Data
Oct. 8, 2004 (FR) 0410648

(51) Int. Cl. C12Q 1/68; C07H 21/04; C12P 19/34

(52) U.S. Cl. 435/6; 536/24.33; 435/91.1

(57) ABSTRACT

The invention relates to polynucleotides and primers flanking a variable region of the intron of the chloroplast gene trnL of plant materials for detecting and identifying plant species. The invention also relates to methods for detecting and identifying plant species in complex or degraded mixtures.

The present invention relates to oligonucleotides and to their use as universal primers for detecting and identifying plant species, in particular in complex or degraded substrates.

Various methods for identifying plants based on analysis of the genome are known, but none make it possible, for the moment, to work on degraded and/or complex substrates.

Genetic fingerprinting methods are thus based on the analysis of the complete genome. The objective of these methods is to provide a genetic fingerprint specific to each individual (identification of the individual and not of the species). However, although this is not the initial objective, they can also make it possible, to a certain extent, to identify the species. However, these methods require that DNA be obtained which is of good quality (not degraded) and is not mixed with exogenous DNAs (originating from other organisms). As a result, it is impossible to use these approaches for identifying plants in degraded or complex substrates.

By way of example of a genetic fingerprinting method, mention may be made of the AFLP and DArT methods.

The AFLP “Amplified Fragment Length Polymorphism” method is currently very widely used both in population genetics and in genetic mapping (Vos P, Hogers R, Bleeker M, Reijans M, van de Lee T, Homes M, Frijters A, Pot J, Peleman J, Kuiper M, Zabeau M (1995) AFLP: a new technique for DNA fingerprinting. Nucleic Acids Research, 23, 4407-4414; U.S. Pat. No. 6,045,994). It is based on a digestion/ligation of genomic DNA, followed by two successive amplifications using specific PCR primers so as to simplify the genome in order to make it analyzable by electrophoresis. It requires DNA of very good quality, and in sufficient amount (several hundred nanograms of DNA in general). It is absolutely impossible to use this approach in a relevant manner for the analysis of degraded and complex substrates.

The DArT “Diversity Array Technology” method is based on a very similar approach (digestion/ligation then amplification) but differs by virtue of the method of analysis (hybridation) (Jaccoud D, Peng K, Feinstein D, Kilian A (2001) Diversity arrays: a solid state technology for sequence information independent genotyping. Nucleic Acids Research, 29, e25; U.S. Pat. No. 6,713,258). It is also impossible to use this approach on degraded and complex substrates.

Other methods are based on amplification and sequencing. From a theoretical point of view, the sequencing of a sufficiently long (several hundred base pairs) homologous region has the potential to allow the identification of the species in plants. Such a region must be framed by very conserved zones that allow universal primers to be designed for the amplification. Nuclear DNA is not very suitable since access to it is very difficult or even impossible in the case of degraded substrates. However, as regards nuclear DNA, ITSs (ribosomal DNA Internal Transcripted Spacers) have been used for detecting and identifying plants. Universal primers have been described in fungi and have been found to also function in plants (White T J, Bruns T, Lee S, Taylor J (1990) Amplification and direct sequencing of fungal ribosomal RNA genes for phylogenetics in: PCR protocols, a guide to methods and applications (eds. Innis M A, Gelfand D H, Sninski J J, White T J), pp. 315-322. Academic Press, San Diego, Calif.). As a result, this region of a few hundred base pairs has been used to determine phylogenies between close species in plants (Baldwin B G (1992) Phylogenetic utility of the internal transcribed spacers of nuclear ribosomal DNA in plants: an example from the Compositae. Molecular Phylogenetics and Evolution, 1, 3-16; Gielly L, Yuan Y-M, Küpfer P, Taberlet P (1996) Phylogenetic use of noncoding regions in the genus Gentiana L.: chloroplast trnL (UAA) intron versus nuclear ribosomal internal transcribed spacer sequences. Molecular Phylogenetics and Evolution, 6, 460-466) and to identify the species (see, for example, Linder C, Moore L, Jackson R (2000) A universal molecular method for identifying underground plant parts to species. Molecular Ecology, 9, 1549-1559 or the website of the company “Bioprofiles”: http://www.bioprofiles.co.uk/). However, this region has drawbacks. Firstly, it is too long to be used in the case of highly degraded substrates; in addition, it involves nuclear sequences that are admittedly repeated, but less so than those present in the chloroplast DNA. Secondly, the primers can amplify several types of sequence within the same species. Finally, the primers are not really universal and it can be difficult to obtain an amplification in certain species.

On the other hand, mitochondrial DNA and chloroplast DNA are present in a highly repeated manner in each cell (several hundred copies). This means that they represent a target for amplification which is much more accessible in the case of degraded substrates. Mitochondrial DNA is, however, not variable enough in plants.

Thus, several articles describe universal primers that target various regions of chloroplast DNA (Taberlet P, Gielly L, Pautou G, Bouvet J (1991) Universal primers for amplification of three non-coding regions of chloroplast DNA. Plant Molecular Biology, 17, 1105-1109; Demesure B, Sodzi N, Petit R J (1995) A set of universal primers for amplification of polymorphic non-coding regions of mitochodrial and chloroplast DNA in plants. Molecular Ecology, 4, 129-131; Dumolin-Lapègue S, Pemonge M-H, Petit R J (1996) An enlarged set of consensus primers for the study of organelle DNA in plants. Molecular Ecology, 5, 393-397; and Hamilton M B (1999) Four primer pairs for the amplification of chloroplast intergenic regions with intraspecific variation. Molecular Ecology, 8, 521-523). Some of them have been widely used for amplifying and sequencing variable regions of chloroplast DNA. They are mainly c and d primers (Taberlet P, Gielly L, Pautou G, Bouvet J (1991) Universal primers for amplification of three non-coding regions of chloroplast DNA. Plant Molecular Biology, 17, 1105-1109) which amplify the intron of the gene of the transfer RNA for leucine, codon UAA (trnL UAA). Currently, several thousand sequences of this intron are available in the public databases (GenBank). The intron of the trnL gene (UAA) is very variable, but also has conserved parts related to the fact that it can constitute secondary structures (Simon D, Fewer D, Friedl T, Bhattacharya D (2003) Phylogeny and self-splicing ability of the plastid tRNA-Leu group I intron. Journal of Molecular Evolution, 57, 710-720). However, these c and d primers amplify regions that are too long to be used on degraded substrates.

Another solution for specific identification has been proposed by Bobowski et al. (Bobowski B, Hole D, Wolf P, Bryant L (1999) Identification of roots of woody species using polymerase chain reaction (PCR) and restricted fragment length polymorphism (RFLP) analysis. Molecular Ecology, 8, 485-491): amplification of the rbcL gene using universal primers, and characterization of the amplification product by enzymatic digestion followed by gel migration (the product could also be characterized by direct sequencing).

However, all these primers amplify regions that are too long to be used on degraded substrates. This is the reason for which other primers were identified by Poinar et al. (Poinar H N, Hofreiter M, Spaulding W G, Martin P S, Stankiewicz B A, Bland H, Evershed R P, Possnert G, Pääbo S (1998) Molecular coproscopy: Dung and diet of the extinct ground sloth Nothrotheriops shastensis. Science, 281, 402-406) in order to amplify shorter fragments, compatible with the analysis of “fossil” residues (coprolites of an extinct sloth in this case). These authors design primers in the chloroplast rbcL gene. The amplified fragments only just make it possible to identify the family, and these primers are not really universal. Despite this, in the absence of an alternative, Willerslev et al. (Willerslev E, Hansen A J, Binladen J, Brand T B, Gilbert M T P, Shapiro B, Bunce M, Wiuf C, Gilichinsky D A, Cooper A (2003) Diverse plant and animal genetic records from Holocene and Pleistocene sediments. Science, 300, 791-795) have used the primers of Poinar et al. (Poinar H N, Hofreiter M, Spaulding W G, Martin P S, Stankiewicz B A, Bland H, Evershed R P, Possnert G, Pääbo S (1998) Molecular coproscopy: Dung and diet of the extinct ground sloth Nothrotheriops shastensis. Science, 281, 402-406) in their analysis of the DNA extracted from permafrost (frozen soil). Their objective was to characterize the plant DNAs still present in soils. Here also, only the families could be identified.

To summarize, either the systems proposed for the moment are based on sequences that are too long to be effective in the analysis of degraded substrates, or the degree of variability of the short fragments is not high enough to be really useful in the identification of plants. Regions that are both sufficiently short and sufficiently variable are rare and none has been characterized.

In order to remedy the drawbacks of the prior art, the present invention proposes novel oligonucleotides and their use as universal primers for detecting and identifying plant species.

The oligonucleotides of the present invention make it possible to amplify a very short but also very variable region of the intron of the trnL (UAA) gene of chloroplast DNA.

A first advantage of the present invention is that the oligonucleotides and the methods of the present invention make it possible to detect and identify plants in complex or degraded substrates such as substrates that have been transformed (by heat, lyophilization, etc.) since the region amplified is both short and very variable.

Another advantage of the present invention is that the region amplified is not only very variable between plant species, but also has very conserved flanking regions that allow amplification of the region of interest in various plant species using universal primers.

Another advantage of the present invention is that the trnL (UAA) gene intron is one of the rare chloroplast sequences for which several thousand sequences are available in databases such as GenBank (http://www.ncbi.nim.nih.gov). The analysis of the variable region amplified using the universal primers therefore makes it possible to identify the corresponding plant species by referring to the sequences available in the databases.

Methods for identifying plants in complex or degraded substrates are of great value. Mention will, for example, be made of applications in the agrofoods industry where, for example, the adherence to traceability criteria means that the development of new analytical methods for identifying the detailed composition of plant species in food preparations is obligatory.

### DESCRIPTION OF THE INVENTION

A first subject of the present invention is a pair of oligonucleotides in which the first oligonucleotide hybridizes to the SEQ ID No. 68 sequence and the second oligonucleotide hybridizes to the SEQ ID No. 69 sequence under stringency conditions which are sufficient for the selective amplification of a variable region of the intron of the trnL chloroplast gene of tobacco, whose sequence is represented at SEQ ID No. 3.

Another subject of the present invention is a pair of oligonucleotides in which the first oligonucleotide hybridizes to the SEQ ID No. 68 sequence and the second oligonucleotide hybridizes to the SEQ ID No. 69 sequence under stringency conditions which are sufficient for the selective amplification of a variable region of the intron of the trnL chloroplast gene of plants whose sequence is represented at SEQ ID Nos. 24-67.

Typically, the hybridization occurs at 55° C. in an amplification buffer comprising 2 mM MgCl₂.

In a preferred embodiment of the invention, the first oligonucleotide is chosen from the group comprising SEQ ID Nos. 1, 4-15 and the second oligonucleotide is chosen from the group comprising SEQ ID Nos. 2, 16-23.

The invention also relates to oligonucleotides whose sequence is chosen from the group comprising SEQ ID No. 1, SEQ ID No. 2, SEQ ID Nos. 4-15 and SEQ ID Nos. 16-23.

The invention also relates to polynucleotides whose sequence is chosen from the group comprising SEQ ID Nos. 24-67.

In an advantageous embodiment of the invention, the pairs of oligonucleotides, the oligonucleotides and the polynucleotides according to the invention are immobilized on a solid support.

Another subject of the present invention is a method for amplifying a variable region of chloroplast DNA of plants, comprising the following steps:
a) a sample including plant genomic DNA is provided;
b) a variable region of chloroplast DNA is amplified with a pair of oligonucleotides according to the invention, or with at least one oligonucleotide according to the invention.

In a specific embodiment, at step b), the variable region of amplified chloroplast DNA is a polynucleotide whose sequence is chosen from the group comprising SEQ ID Nos. 24-67.

In another specific embodiment, the method for amplifying a variable region of chloroplast DNA of plants according to the invention comprises a step consisting of extraction of the chloroplast DNA before the amplification step b).

Preferably, the variable region of chloroplast DNA is amplified by means of a polymerase chain reaction (PCR).

The invention also relates to a method for detecting a plant species in a sample, comprising the following steps:
a) a sample suspected of containing a plant species is provided;
b) an amplification reaction is carried out with a pair of oligonucleotides according to the invention, or with at least one oligonucleotide according to the invention;
c) detection of whether an amplification product proving the presence of a plant species in the sample is obtained.

In a specific embodiment, the amplification product is a polynucleotide whose sequence is chosen from the group comprising SEQ ID Nos. 24-67.

In another embodiment, the method for detecting a plant species in a sample according to the invention comprises a step consisting of the extraction of the DNA before the amplification step b).

Preferably, a polymerase chain reaction (PCR) is carried out.

The invention also relates to a method for identifying a plant species in a sample, comprising the following steps:
a) a sample suspected of containing a plant species is provided;
b) an amplification reaction is carried out with a pair of oligonucleotides according to the invention, or with at least one oligonucleotide according to the invention;
c) the amplification product thus obtained is analyzed to identify the plant species contained in the sample.

In a specific embodiment, the amplification product is a polynucleotide whose sequence is chosen from the group comprising SEQ ID Nos. 24-67.

In one embodiment, at step c), the sequence of the amplification product is determined for identifying the plant species contained in the sample.

In another embodiment, at step c), the amplification product is hybridized with at least one reference plant sequence for identifying the plant species contained in the sample.

Preferably, the reference sequence is chosen from the group comprising SEQ ID Nos. 3 and 24-67.

In another embodiment, at step c), the amplification product is analyzed by electrophoresis for identifying the plant species contained in the sample.

The invention also relates to the use of the variable region of the intron of the trnL chloroplast gene of plants corresponding to positions 49425 to 49466 of the chloroplast DNA of tobacco for detecting and identifying plant species.

In a specific embodiment, the variable region of the intron of the trnL chloroplast gene of plants is a polynucleotide whose sequence is chosen from the group comprising SEQ ID Nos. 24-67.

The present invention relates to polynucleotides derived from two very conserved regions of chloroplast DNA of plants. These polynucleotides derived from regions whose sequence is very conserved throughout the plant kingdom, in particular in angiosperms and gymnosperms, can be used as universal primers for amplifying or sequencing chloroplast DNA of plants.

In addition, it has been found, particularly advantageously, that the conserved regions from which the polynucleotides of the present invention are derived flank a region of chloroplast DNA which is both short and very variable. The variability of this region between plant species can therefore be used for distinguishing and identifying plant species.

According to the present invention, the term “polynucleotide” is intended to mean a single-stranded nucleotide chain or the chain complementary thereto, or a double-stranded nucleotide chain, that may be of DNA or RNA type. The polynucleotides of the invention are preferably of DNA type, in particular double-stranded DNA.

The term “polynucleotide” also denotes oligonucleotides and polynucleotides that have been modified. Typically, the modified polynucleotides can contain modified nucleotides. Alternatively, these modified polynucleotides are polynucleotides conjugated to binding reagents (biotin, for example) or to labeled reagents (fluorescent labels, for example). Conventionally, the binding reagents for the labeled reagents conjugated to the polynucleotides facilitate the purification or the detection of these polynucleotides.

According to the invention, the term “oligonucleotide” is intended to mean a polynucleotide consisting of a short sequence of nucleotides, the number of which varies from one to a few tens, but is generally less than 100 bases. The term “polynucleotide” therefore also denotes oligonucleotides.

The term “primer” is intended to mean a short oligonucleotide sequence which, when hybridized with a nucleic acid template, allows a polymerase to initiate the synthesis of a new DNA strand. The strand produced from the primer is complementary to the strand used as template.

Advantageously, the polynucleotides of the present invention can be immobilized on a solid support. Solid supports suitable for the immobilization of polynucleotides or oligonucleotides, in particular for the fabrication of DNA chips, are known. Many varieties of DNA chips exist, which differ by virtue of the type of support used, the nature, the density and the method of attachment or of synthesis of the nucleotide sequences on the support, and the reading conditions. These techniques are known to those skilled in the art. The term “solid support” is also intended to mean supports of microsphere type, such as the FLEXMAP™ products from the company LUMINEX® and the LiquidChip™ products from the company QIAGEN®.

In general, the polynucleotides of the present invention are isolated or purified form their natural environment. Preferably, the polynucleotides of the present invention can be prepared by the conventional molecular biology techniques as described by Sambrook et al. (Molecular Cloning: A Laboratory Manual, 1989) or by chemical synthesis.

The term “plant species” is intended to mean any live organism that is part of the plant kingdom.

The invention also relates to a pair of oligonucleotides in which the first oligonucleotide hybridizes to a first very conserved region of chloroplast DNA and the second oligonucleotide hybridizes to a second very conserved region of chloroplast DNA under stringency conditions which are sufficient for the selective amplification of a variable region of the intron of the trnL chloroplast gene of plants. The sequence of the first conserved region corresponds to the sequence of the primer g (SEQ ID No. 1) and of the sequence complementary thereto (SEQ ID No. 68). The sequence of the second conserved region corresponds to the sequence of the primer h (SEQ ID No. 2) and of the sequence complementary thereto (SEQ ID No. 69).

In tobacco, which can be used as reference plant species, the pairs of oligonucleotides of the present invention allow the selective amplification of the variable region of the intron of the trnL chloroplast gene of tobacco, whose sequence is represented at SEQ ID No. 3.

The sequences of the pairs of oligonucleotides of the present invention are chosen in such a way that the first oligonucleotide hybridizes to SEQ ID No. 68 and the second oligonucleotide hybridizes to SEQ ID No. 69 under stringency conditions which are sufficient for the selective amplification of the variable region of the intron of the trnL chloroplast gene of plants.

Those skilled in the art are aware of the DNA amplification reactions and the stringency conditions for selective amplification, and in particular the hybridization temperature conditions and hybridization buffer composition conditions.

Those skilled in the art may therefore readily define different variants of the primers g (SEQ ID No. 1) and h (SEQ ID No. 2) using routine techniques. These variants hybridize to the reference sequences and allow the selective amplification of the variable region of interest of chloroplast DNA. Certain possible variants of the primer g are represented at SEQ ID Nos. 4-15 and certain possible variants of the primer h are represented at SEQ ID Nos. 16-23. Usually, the sequence variations are introduced at the 5′ end of the oligonucleotides so as not to compromise the amplification reaction. Conventionally, it is possible, for example, to introduce additional nucleotides at the 5′ end of the oligonucleotides.

According to the invention, the term “hybridize” is intended to mean the sequences which hybridize with the reference sequence at a level significantly greater than the background noise. The level of the signal generated by the interaction between the sequence capable of selectively hybridizing and the reference sequences is generally 10 times, preferably 100 times, more intense than that of the interaction of the other DNA sequences which generate the background noise. The stringent hybridization conditions for selective hybridization are well known to those skilled in the art. In general, the hybridization and washing temperature is at least 5° C. below the Tm of the reference sequence at a given pH and for a given ionic strength. Typically, the hybridization temperature is at least 30° C. for a polynucleotide of 15 to 50 nucleotides and at least 60° C. for a polynucleotide of more than 50 nucleotides. By way of example, the hybridization is carried out in the following buffer: 6×SSC, 50 mM Tris-HCl (pH 7.5), 1 mM EDTA, 0.02% PVP, 0.02% Ficoll, 0.02% BSA, 500 μg/ml denatured salmon sperm DNA. The washes are, for example, carried out successively at low stringency in a 2×SSC, 0.1% SDS buffer, at medium stringency in a 0.5×SSC, 01% SDS buffer, and at high stringency in a 0.1×SSC, 0.1% SDS buffer. The hybridization can of course be carried out according to other usual methods well known to those skilled in the art (see, in particular, Sambrook et al., Molecular Cloning: A Laboratory Manual, 1989).

Preferably, the polynucleotides which hybridize selectively to a reference polynucleotide conserve the function of the reference sequence. In the present invention, the function of the polynucleotides is the amplification of a variable region of chloroplast DNA.

The term “stringency” is intended to mean the strictness of the operating conditions (in particular the temperature and the ionic strength) under which a molecular hybridization takes place.

The term “amplification” is intended to mean any in vitro enzymatic amplification of a defined DNA sequence.

Usually, the amplification comprises successive amplification cycles (generally from 20 to 40), which are themselves composed of three phases: after a DNA denaturation step (separation of the two strands of the double helix), the positioning of the primers (specifically chosen short oligonucleotide sequences) opposite the sequences complementary thereto, on the DNA strands, and the binding thereof to these targets, constitutes the second phase of the method (hybridization). The extension phase involves an enzyme, DNA polymerase, which synthesizes, from the primers, the strand complementary to that which served as a template. The repetition of this cycle results in the exponential amplification of the DNA fragment.

The invention also relates to a method for amplifying a variable region of chloroplast DNA of plants using the polynucleotides, the oligonucleotides and/or the pairs of oligonucleotides according to the invention.

The methods for amplifying a DNA sequence are well known to those skilled in the art and widely described in the literature. Mention will be made of the polymerase chain reaction (PCR), but any type of amplification reaction can be used in the methods according to the invention.

Given that the sequences of the polynucleotides according to the present invention are highly conserved throughout the plant kingdom, these polynucleotides can be used for the detection of plant species.

The term “detection” is intended to mean the determination of the presence of a plant species in a sample, but also the measurement and the quantification of a plant species in a sample.

Using the polynucleotides of the present invention, it is now possible to amplify a very variable region of chloroplast DNA. The sequence of this region differs from one plant species to the other such that each sequence is specific for a species or for a small number of very close species. Once the variable region has been amplified, its sequence is analyzed in order to identify the plant species. The analysis can be carried out by various methods well known to those skilled in the art. It may be complete or partial sequencing followed by a comparison with known sequences. Alternatively, it may involve the determination of the degree of homology with known sequences (reference sequences) using hybridization techniques, for example. Another possibility is analysis by electrophoresis and then comparison with reference sequences. The methods according to the present invention therefore make it possible to determine the identity of a plant species present in a sample.

The term “sample” in an analytical procedure is intended to mean the substance to be measured. In the present invention, the sample usually comprises an organic substance suspected of containing a plant species. Advantageously, the methods of the present invention allow the analysis of samples consisting of material that is decomposing or has been degraded by heating, lyophilization or freezing or by any other treatment that results in degradation of the DNA. The methods of the present invention thus allow the detection of plant species in transformed foods, for example. Another application of the methods of the present invention is the detection of plant species in substrates derived from frozen soils (permafrost) or in fossilized residues.

The sample can undergo a treatment before the amplification reaction using the polynucleotides of the invention is carried out. Typically, it may be a DNA extraction step according to routine techniques well known to those skilled in the art.

The term “extraction” is intended to mean the process consisting in extracting a substance from a medium using, for example, a solvent, or by any other physicochemical method.

The invention also relates to the use of the variable region of the intron of the trnL chloroplast gene of plants corresponding to positions 49425 to 49466 of the chloroplast DNA of tobacco for detecting and identifying plant species.

Based on the tobacco reference sequence (Shinozaki K, Ohme M, Tanaka M, Wakasugi T, Hayashida N, Matsubayashi T, Zaita N, Chunwongse J, Obokata J, Yamaguchi-Sinozaki K, Ohto C, Torazawa K, Ment B, Sugita M, Deno H, Kamogashira T, Yamada K, Kusuda J, Takaiwa F, Kato A, Tohdoh N, Shimada H (1986) The complete nucleotide sequence of the tobacco chloroplast genome. Plant Molecular Biology Reporter, 4, 110-147) and on the positions of the variable region on this reference sequence, those skilled in the art can identify the corresponding sequences in other plant species using routine techniques.

### DESCRIPTION OF THE SEQUENCE LISTING

### SEQ ID No. 1: Primer g.

### SEQ ID No. 2: Primer h.

SEQ ID No. 3: Amplified variable sequence of Nicotiana tabacum.
SEQ ID No. 4-15: Variants of the primer g.
SEQ ID No. 16-23: Variants of the primer h.
SEQ ID No. 24-67: Amplified variable sequence of various plant species.
SEQ ID No. 68: Sequence of the region complementary to the primer g.
SEQ ID No. 69: Sequence of the region complementary to the primer h.

### DESCRIPTION OF THE FIGURES

FIG. 1: Location of the zone studied and of the universal primers on the tobacco chloroplast DNA sequence (Shinozaki K, Ohme M, Tanaka M, Wakasugi T, Hayashida N, Matsubayashi T, Zaita N, Chunwongse J, Obokata J, Yamaguchi-Sinozaki K, Ohto C, Torazawa K, Ment B, Sugita M, Deno H, Kamogashira T, Yamada K, Kusuda J, Takaiwa F, Kato A, Tohdoh N, Shimada H (1986) The complete nucleotide sequence of the tobacco chloroplast genome. Plant Molecular Biology Reporter, 4, 110-147); c and d represent the primers defined by Taberlet et al. (Taberlet P, Gielly L, Pautou G, Bouvet J (1991) Universal primers for amplification of three non-coding regions of chloroplast DNA. Plant Molecular Biology, 17, 1105-1109), g and h represent the universal primers defined in the context of this patent application.

FIG. 2: Examples of amplifications obtained with the primers g and h, using extracts of DNA originating from degraded substrates. 1, cooked potato; 2, cooked pasta; 3 and 4, freeze-dried packet soup; 5, negative control for the extraction (amplification reaction using an extraction without substrate); 6, negative control for amplification (amplification reaction without DNA extract); 7, positive control (Cyclamen DNA); M, molecular weight marker. It is interesting to note that the fragment corresponding to the cooked potato (79 bp) is shorter than that corresponding to the cooked pasta and therefore to wheat (92 bp).

FIG. 3: Experiment comparing the efficiency of the primers c-d (lanes 1-4) and g-h (lanes 5-8) for the amplification of DNA extracted from a degraded substrate (breadcrumbs). M, molecular weight marker. 1 and 5, DNA extracted from breadcrumbs. 2 and 6, extraction control. 3 and 7, amplification control. 4 and 8, positive control.

FIG. 4: FIG. 4 shows, in schematic form, the general approach which could be applied for identifying plants using certain embodiments of the invention. The first step consists in extracting the DNA from the substrate. The second step is the amplification of the extract using the PCR (Polymerase Chain Reaction) method. The analysis of the amplification product constitutes the third step. Four alternative solutions are shown. The first analytical possibility concerns only simple substrates (a single plant species present) and consists in directly sequencing the amplification product with conventional methods. The second and third possibilities are reserved for complex substrates containing a mixture of plants. The analysis is either carried out after cloning the amplification product and then sequencing several clones (see Table 4 for an example of a result), or after hybridization on a support with the potential target sequences (method not illustrated, which involves prior knowledge of the species that may be present). A final analytical possibility consists in characterizing the amplification products by electrophoresis (either denaturing, or nondenaturing of the SSCP type). The latter possibility can equally be used for simple substrates (a single plant species present) or for substrates containing a mixture.

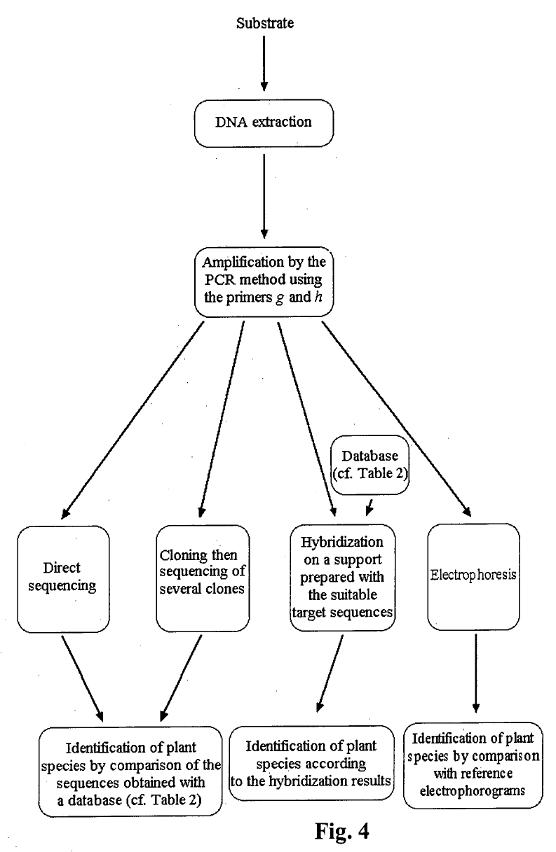

As regards analysis by direct sequencing, the PCR conditions used for the detection by electrophoresis are the following:

### EXAMPLES

### 1) Direct Sequencing

As regards analysis by direct sequencing, the PCR conditions used for the detection by electrophoresis are the following:

a) Amplification conditions for detection with primer g labeled with a fluorochrome:

(i) final volume: 25 μl

(ii) MgCl₂: 2 mM

(iii) dNTP: 0.2 mM each

(iv) Primers: 1 μM each

(v) Taq polymerase (Amplitaq Gold, Perkin Elmer): 1 unit

(vi) BSA: 0.2 μl per tube

(vii) Volume of DNA extract used: 2.5 μl

(viii) Initial denaturation of 10 nm at 95° C.

(ix) Number of cycles: 35 (to be adjusted according to the extract)

(x) Denaturation: 30 s at 95° C., hybridization: 30 s at 55° C., no extension step.

These conditions are aimed at reducing the “+A” artifact which hinders the interpretation of the results.
b) Amplification conditions for detection with primer h labeled with a fluorochrome:

(i) final volume: 25 μl

(ii) MgCl₂: 2 mM

(iii) dNTP: 0.2 mM each

(iv) Primers: 1 μM each

(v) Taq polymerase (Amplitaq Gold, Perkin Elmer): 1 unit

(vi) BSA: 0.2 μl per tube

(vii) Volume of DNA extract used: 2.5 μl

(viii) Initial denaturation of 10 nm at 95° C.

(ix) Number of cycles: 35 (to be adjusted according to the extract)

(x) Denaturation: 30 s at 95° C., hybridization: 30 s at 55° C., extension: 60 s at 72° C.

(xi) Final extension: 90 minutes at 72° C.

These conditions are aimed at promoting the “+A” artifact in order to facilitate the interpretation of the results.

### 2) Universal Primers

Table 1 represents the sequences of the universal primers used to amplify the variable region of the chloroplast DNA for identifying the plants after extraction and amplification from degraded substrates. The positions of the 3′ base on the tobacco reference sequence are indicated in the table (Shinozaki K, Ohme M, Tanaka M, Wakasugi T, Hayashida N, Matsubayashi T, Zaita N, Chunwongse J, Obokata J, Yamaguchi-Sinozaki K, Ohto C, Torazawa K, Ment B, Sugita M, Deno H, Kamogashira T, Yamada K, Kusuda J, Takaiwa F, Kato A, Tohdoh N, Shimada H (1986) The complete nucleotide sequence of the tobacco chloroplast genome. Plant Molecular Biology Reporter, 4, 110-147).

TABLE 1

| | | Position of the 3′ base |
|---|---|---|
| Primer | Sequence | on the tobacco sequence |
| g | 5′GGGCAATCCTGAGCC | 49425 |
| | AA 3′ | |
| h | 5′CCATTGAGTCTCTGC | 49466 |
| | ACCTATC 3′ | |

The alignment of these two flanking regions shows (Tables 1 and 2) that it is possible to define primers which are universal in higher plants (angiosperms and gymnosperms). After having aligned several hundred trnL (UM) intron sequences, the few sequence variations observed in the zone where we defined the primers demonstrate that the latter are really universal (see Table 2). In fact, the difference observed with the sequence of the primer involves at most a single mismatch that does not in any way affect the last three bases on the 3′ side of the primer. As a result, it is possible to predict with certainty that the primers g and h are universal.

### 3) Variability of the Amplified Region

Table 2 shows the variations of the zone amplified by the primers g and h for various plant species that are part of the composition of foods (see also Table 3). These sequences were either imported from GenBank (public DNA sequence database) or were produced in our laboratory.

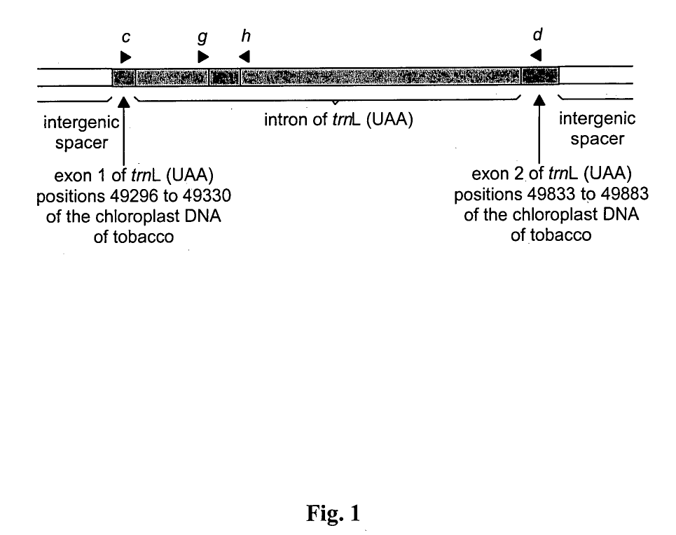

Two very conserved regions frame a very variable part of a length of approximately 20 to 100 base pairs (FIG. 1). Such a region therefore represents the ideal target for identifying plants from degraded substrates (under these conditions, it is often difficult to obtain amplification products for fragments greater than 120 base pairs in length). We did not find other regions that met these criteria. It therefore appears that the system that we are proposing is unique.

Table 2 represents the sequence alignment showing, firstly, the zone on which the universal primers were defined and, secondly, the variability of the amplified region. The nucleotides underlined in the regions corresponding to the primers indicate the mismatches with the universal primers g and h. As regards the amplified region, the underlining indicates identical sequences.

TABLE 2

| | Sequence of the | | Sequence of the |
|---|---|---|---|
| | region | | region |
| Scientific | corresponding to | | corresponding to |
| name | the primer g | Sequence of the amplified region | the primer h |
| Theobroa | GGGCAATCCTG | ATCCTATTATTTTATTATTTTACGAAAC | GATAGGTGCA |
| cacao | AGCCAA | TAAACAAAGGTTCAGCAAGCGAGAAT | GAGACTCAAT |
| | | AATAATAAAAAAAG | GG |
| Beta vulgaris | GGGCAATCCTG | CTCCTTTTTTCAAAAGAAAAAAAATAA | GATAGGTGCA |
| | AGCCAA | GGATTCCGAAAACAAGAATAAAAAAA | GAGACTCAAA |
| | | AAG | GG |
| Castanea | GGGCAATCCTG | ATCCTATTTTACGAAAACAAATAAGGG | GATAGGTGCA |
| sativa | AGCCAA | TTCAGAAGAAAGCGAGAATAAAAAAA | GAGACTCAAT |
| | | AG | GG |
| Cannabis | GGGCAATCCTG | ATCCGGTTTTCTGAAAACAAACAAGGA | GATAGGTGCA |
| sativa | AGCCAA | TTCAGAAAGCAATAATAAAAAAGAAT | GAGACTCAAT |
| | | AG | GG |
| Cicer arietinum | GGGCAATCCTG | ATCCTGCTTTCGGAAAACAAACAAAAA | GATAGGTGCA |
| | AGCCAA | AAGTTCAGAAAGTTAAAATCAAAAAA | GAGACTCAAT |
| | | G | GG |
| Saccharum | GGGCAATCCTG | ATCCCCTTTTTTGAAAAAACAAGTGGT | GATAGGTGCA |
| officinarum | AGCCAA | TCTCAAACTAGAACCCAAAGGAAAAG | GAGACTCAAT |
| | | | GG |
| Asparagus | GGGCAATCCTG | ATCTTTATGTTTAGAAAAACAAGGGTT | GATAGGTGCA |
| officinalis | AGCCAA | TTAATTTAAAAACTAGAAGAAAAAGG | GAGACTCAAT |
| | | | GG |
| Triticum | GGGCAATCCTG | ATCCGTGTTTTGAGAAAACAAGGGGTT | GATAGGTGCA |
| aestivum | AGCCAA | CTCGAACTAGAATACAAAGGAAAAG | GAGAGTCAAT |
| | | | GG |
| Secale cereale | GGGCAATCCTG | ATCCGTGTTTGAGAAAACAAGGGGTT | GATAGGTGCA |
| | AGCCAA | CTCGAACTAGAATACAAAGGAAAAG | GAGACTCAAT |
| | | | GG |
| Oryza sativa | GGGCAATCCTG | ATCCATGTTTTGAGAAAACAAGCGGT | GATAGGTGCA |
| | AGCCAA | CTCGAACTAGAACCCAAAGGAAAAG | GAGACTCAAT |
| | | | GG |
| Panicum | GGGCAATCCTG | ATCCCTTTTTGAAAAAACAAGTGGTT | GATAGGTGCA |
| miliaceum | AGCCAA | CTCAAACTAGAACCCAAAGGAAAAG | GAGACTCAAT |
| | | | GG |
| Ribes aureum | GGGCAATCCTG | ATCCTGTTTTACAAACAAAACACAAGA | GATAGGTGCA |
| | AGCCAA | GTTCACAAAGAGAGAATAAAAAAAG | GAGACTCAAT |
| | | | GG |
| Fragaria vesca | GGGCAATCCTG | ATCCGGTTTTATGAAAACAAACAAGGG | GATAGGTGCA |
| | AGCCAA | TTTCAGAAAGCGAGAATAAATAAAG | GAGACTCAAT |
| | | | GG |
| Citrus x | GGGTAATCCTG | ATCCTCTTCTCTTTTCCAAGAACAAAC | GATAGGTGCA |
| paradisi | AGCCAA | AGGGGTTCAGAAAGCGAAAAAGGGG | GAGACTCAAT |
| | | | GG |
| Triphasia | GGGTAATCCTG | ATCCTCTTCTCTTTTCCAAGAACAAAC | GATAGGTGCA |
| trifolia | AGCCAA | AGGGGTTCAGAAAGCGAAAAAGGGG | GAGACTCAAT |
| | | | GG |
| Vitis vinifera | GGGCAATCCTG | ATCCTGTTTTCCGAAAACAACCAAGGG | GATAGGTGCA |
| | AGCCAA | TTCAGAAAACGATAATAAAAAAAG | GAGACTCAAT |
| | | | GG |
| Prunus persica | GGGCGATCCTG | ATCCTGTTTTATTAAAACAAACAAGGG | GATAGGTGCA |
| | AGCCAA | TTTCATAAACCGAGAATAAAAAAG | GAGACTCAAT |
| | | | GG |
| Prunus | GGGCGATCCTG | ATCCTGTTTTATTAAAACAAACAAGGG | GATAGGTGCA |
| armeriana | AGCCAA | TTTCATAAACCGAGAATAAAAAAG | GAGACTCAAT |
| | | | GG |
| Prunus cerasus | GGGCGATCCTG | ATCCTGTTTTATTAAAACAAACAAGGG | GATAGGTGCA |
| | AGCCAA | TTTCATAAACCGAGAATAAAAAAG | GAGACTCAAT |
| | | | GG |
| Actinidia | GGGCAATCCTG | ATCCTTTTTTTCGAAAACAAACAAAGA | GATAGGTGCA |
| chinensis | AGCCAA | TTCAGAAAGCGAAAATAAAACAAG | GAGACTCAAT |
| | | | GG |
| Zea mais | GGGCAATCCTG | ATCCCTTTTTTGAAAAACAAGTGGTTC | GATAGGTGCA |
| | AGCCAA | TCAAACTAGAACCCAAAGGAAAAG | GAGACTCAAT |
| | | | GG |
| Pisum sativum | GGGCAATCCTG | ATCCTTCTTTCTGAAAACAAATAAAAG | GATAGGTGCA |
| | AGCCAA | TTCAGAAAGTGAAAATCAAAAAAG | GAGACTCAAT |
| | | | GG |
| Phaseolus | GGGCAATCCTG | ATCCCGTTTTCTGAAAAAAAGAAAAAT | GATAGGTGCA |
| vulgaris | AGCCAA | TCAGAAAGTGATAATAAAAAAGG | GAGACTCTAT |
| | | | GG |
| Sorghum | GGGCAATCCTG | ATCCACTTTTTTCAAAAAAGTGGTTCT | GATAGGTGCA |
| halepense | AGCCAA | CAAACTAGAACCCAAAGGAAAAG | GAGACTCAAT |
| | | | GG |
| Cynara | GGGCAATCCTG | ATCACGTTTTCCGAAACTAAACAAAGG | GATAGGTGCA |
| cardunculus | AGCCAA | TTCAGAAAGCGAAAATCAAAAAG | GAGACTCGAT |
| | | | GG |
| Arctium lappa | GGGCAATCCTG | ATCACGTTTTCCGAAAACAAACAAAGG | GATAGGTGCA |
| | AGCCAA | TTCAGAAAGCGAAAATAAAAAAG | GAGACTCGAT |
| | | | GG |
| Lactuca sativa | GGGCAATCCTG | ATCACGTTTTCCGAAAACAAACAAAGG | GATAGGTGCA |
| | AGCCAA | TTCAGAAAGCGAAAATAAAAAAG | GAGACTCGAT |
| | | | GG |
| Helianthus | GGGCAATCCTG | ATCACGTTTTCCGAAAACAAACAAAGG | GATAGGTGCA |
| annus | AGCCAA | TTCAGAAAGCGAAAATAAAAAAG | GAGACTCGAT |
| | | | GG |
| Ficus carica | GGGCAATCCTG | ATCCGGTTTTCTGAAAACAAACAAGGG | GATAGGTGCA |
| | AGCCAA | TTCAGAAAGGCGATAATAAAAAAG | GAGACTCAAT |
| | | | GG |
| Humulus | GGGCAATCCTG | ATCCGGTTTTCTGAAAACAAACAAGGA | GATAGGTGCA |
| lupulus | AGCCAA | TTCAGAAAGCAATAATAAAGGG | GAGACTCAAT |
| | | | GG |
| Avena sativa | GGGCAATCCTG | ATCCGTGTTTTGAGAGGGGGGTTCTCG | GATAGGTGCA |
| | AGCCAA | AACTAGAATACAAAGGAAAAG | GAGAGTCAAT |
| | | | GG |
| Nasturtium | GGGCAATCCTG | ATCCTTGTTTACGCAAACAAACCGGAG | GATAGGTGCA |
| officinale | AGCCAA | TTTAGAAAGCGAGAAAAAAGG | GAGACTCAAT |
| | | | GG |
| Armoracia | GGGCAATCCTG | ATCCTTGTTTACGCGAACAAACCTGAG | GATAGGTGCA |
| rusticana | AGCCAA | TTTAGAAAGCGAGATAAAAGG | GAGACTCAAT |
| | | | GG |
| Hordeum | GGGCAATCCTG | ATCCGTGTTTTGAGAAGGGATTCTCGA | GATAGGTGCA |
| vulgare | AGCCAA | ACTAGAATACAAAGGAAAAG | GAGACTCAAT |
| | | | GG |
| Anthriscus | GGGCAATCCTG | ATCCTATTTTTTCCAAAAACAAACAAA | GATAGGTGCA |
| cerefolium | AGCCAA | GGCCCAGAAGGTGAAAAAAG | GAGACTCAAT |
| | | | GG |
| Allium cepa | GGGCAATCCTG | ATCTTTCTTTTTTGAAAAACAAGGGTTT | GATAGGTGCA |
| | AGCCAA | AAAAAAGAGAATAAAAAAG | GAGACTCAAT |
| | | | GG |
| Allium porum | GGGCAATCCTG | ATCTTTATTTTTGAAAAACAAGGGTT | GATAGGTGCA |
| | AGCCAA | TAAAAAAGAGAATAAAAAAG | GAGACTCAAT |
| | | | GG |
| Carum | GGGCAATCCTG | ATCCTATTTTCCAAAAACAAACAAAGG | GATAGGTGGA |
| petroselinum | AGCCAA | CCCAGAAGGTGAAAAAAG | GAGAGTCAAT |
| | | | GG |
| Solanum | GGGCAATCCTG | ATCCTGTTTTCTGAAAACAAACAAAGG | GATAGGTGCA |
| tuberosum | AGCCAA | TTCAGAAAAAAAG | GAGACTCAAT |
| | | | GG |
| Solanum | GGGCAATCCTG | ATCCTGTTTTCTGAAAACAAACCAAGG | GATAGGTGCA |
| lycopersicum | AGCCAA | TTCAGAAAAAAAG | GAGAGTCAAT |
| | | | GG |
| Solanum | GGGCAATCCTG | ATCCTGTTTTCTCAAAACAAACAAAGG | GATAGGTGCA |
| melongena | AGCCAA | TTCAGAAAAAAAG | GAGAGTCAAT |
| | | | GG |
| Raphanus | GGGCAATCCTG | ATCCTGAGTTACGCGAACAAACCAGA | GATAGGTGCA |
| sativus | AGCCAA | GTTTAGAAAGCGG | GAGACTCAAT |
| | | | GG |
| Brassicaoleraceacapitata | GGGCAATCCTGAGCCAA | 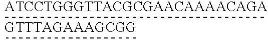 | GATAGGTGCAGAGACTCAATGG |
| Brassicaraparapa | GGGCAATCCTGAGCCAA | 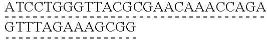 | GATAGGTGCAGAGACTCAATGG |
| Brassica nigra | GGGCAATCCTG | ATCCTGGGTTACGCGAACAAACCAGA | GATAGGTGCA |
| | AGCCAA | GTTTAGAAAGCGG | GAGACTCAAT |
| | | | GG |
| Olea europaea | GGGCAATCCTG | ATCCTGTTTTCCCAAAACAAAGGTTCA | GATAGGTGCA |
| | AGCCAA | GAAAGAAAAAAG | GAGACTCAAT |
| | | | GG |
| Uritaca dioica | GGGCAATCCTG | ATCTGGTGTTATAAAACAAAGCGATAA | GATAGGTGCA |
| | AAACCAA | AAAAAAG | GAGACTCAAC |
| | | | GG |
| Rumex acetosa | GGGCAATCCTG | CTCCTCCTTTCCAAAAGGAAGAATAAA | GATAGGTGCA |
| | AGCCAA | AAAG | GAGACTCAAT |
| | | | GG |

The amplified region shows not only a size variation between the various species, but also a sequence variation. It is interesting to note that the degree of variability makes it possible to identify the vast majority of species consumed. However, close species may, in certain cases, not be discernible. This is the case in our example between wheat and rye, and between cabbage and turnip.

Table 3 represents the common names and origins of the sequences of the foods of Table 2. LECA=sequences produced by the inventors

TABLE 3

| Scientific name | Name of food | Origin |
|---|---|---|
| Theobroa cacao | cocoa | LECA |
| Beta vulgaris | sugar beet | LECA |
| Castanea sativa | sweet chestnut | GenBank: |
| | | AF133653 |
| Cannabis sativa | cannabis | GenBank: |
| | | AF501598 |
| Cicer arietinum | chickpea | GenBank: |
| | | AB117648 |
| Saccharum officinarum | sugar cane | GenBank: |
| | | AY116253 |
| Asparagus officinalis | asparagus | GenBank: |
| | | AJ441164 |
| Triticum aestivum | wheat | GenBank: |
| | | AB042240 |
| Secale cereale | rye | GenBank: |
| | | AF519162 |
| Oryza sativa | rice | GenBank: |
| | | X15901 |
| Panicum miliaceum | millet | GenBank: |
| | | AY142738 |
| Ribes aureum | golden currant | GenBank: |
| | | AF374816 |
| Fragaria vesca | strawberry | LECA |
| Citrus x paradisi | lemon/orange | GenBank: |
| | | AY295277 |
| Triphasia trifolia | limeberry | GenBank: |
| | | AY295297 |
| Vitis vinifera | grape | LECA |
| Prunus persica | peach | GenBank: |
| | | AF348560 |
| Prunus armeriana | apricot | LECA |
| Prunus cerasus | cherry | LECA |
| Actinidia chinensis | kiwi | GenBank: |
| | | AF534655 |
| Zea mais | maize | GenBank: |
| | | NC_001666 |
| Pisum sativum | garden pea | LECA |
| Phaseolus vulgaris | bean | GenBank: |
| | | AY077945 |
| Sorghum halepense | sorghum | GenBank: |
| | | AY116244 |
| Cynara cardunculus | artichoke | GenBank: |
| | | AF129828 |
| Arctium lappa | greater burdock | GenBank: |
| | | AF129824 |
| Lactuca sativa | lettuce | GenBank: |
| | | U82042 |
| Helianthus annuus | sunflower | GenBank: |
| | | U82038 |
| Ficus carica | fig | LECA |
| Humulus lupulus | hops | GenBank: |
| | | AF501599 |
| Avena sativa | oats | GenBank: |
| | | X75695 |
| Nasturtium officinale | cress | GenBank: |
| | | AY122457 |
| Armoracia rusticana | horseradish | GenBank: |
| | | AF079350 |
| Hordeum vulgare | barley | GenBank: |
| | | X74574 |
| Anthriscus cerefolium | chervil | GenBank: |
| | | AF432022 |
| Allium cepa | onion | LECA |
| Allium porum | leek | LECA |
| Carum petroselinum | parsley | LECA |
| Solanum tuberosum | potato | LECA |
| Solarium lycopersicum | tomato | GenBank: |
| | | AY098703 |
| Solanum melongena | aubergine | GenBank: |
| | | AY266240 |
| Raphanus sativus | radish | GenBank: |
| | | AF451576 |
| Brassica oleracea capitata | cabbage | GenBank: |
| | | AF451574 |
| Brassica rapa rapa | turnip | GenBank: |
| | | AF451573 |
| Brassica nigra | black mustard | GenBank: |
| | | AF451579 |
| Olea europaea | olive | LECA |
| Urtica dioica | nettle | GenBank: |
| | | AY208725 |
| Rumex acetosa | sorrel | GenBank: |
| | | AY177334 |

### 4) Examples of Applications to Degraded Substrates

We carried out several experiments which demonstrate clearly the validity of the approach proposed in the present application.

The DNA of several complex and/or transformed substrates was extracted using a conventional extraction kit and by following the manufacturer's instructions (Dneasy Plant Mini Kit, Qiagen). The substrates tested are the following:

(i) sugar cane

(ii) cooked potato

(iii) cooked pasta

(iv) freeze-dried packet soup

For the solid foods, the DNA was extracted from 50 mg of dry weight. The final volume of the DNA extract was recovered in 200 μl.

The amplification was carried out using the primers g and h (Table 1), and the following amplification conditions:

(i) Final volume: 25 μl

(ii) MgCl₂: 2 mM

(iii) dNTP: 0.2 mM each

(iv) Primers: 1 μM each

(v) Taq polymerase (Amplitaq Gold, Perkin Elmer): 1 unit

(vi) BSA: 0.2 μl per tube

(vii) Volume of DNA extract used: 2.5 μl ( 1/80 of the extract)

(viii) Initial denaturation of 10 nm at 95° C.

(ix) Number of cycles: 35 (except for the sugar cane: 50)

(x) Denaturation: 30 s at 95° C., hybridization: 30 s at 55° C., extension: 60 s at 72° C.

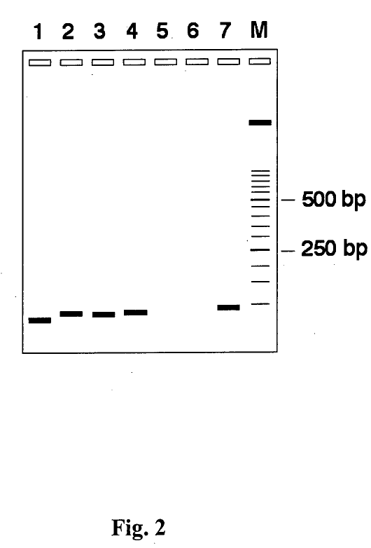

FIG. 2 illustrates an amplification result. The amplification products were then sequenced by direct sequencing on an ABI 3100 capillary automatic sequencer. The sequences obtained for the sugar cane, the cooked potato and the cooked pasta are identical, respectively, to the sequences of sugar cane (Saccharum officinarum), potato (Solanum tuberosum) and wheat (Triticum aestivum). On the other hand, the sequence obtained by direct sequencing for the freeze-dried packet soup is not readable, which indicates that it is a mixture. We therefore cloned the PCR product from the freeze-dried packet soup in order to separate the various molecules. Out of 23 clones sequenced, we obtained 19 clones containing the leek sequence, three clones containing the potato sequence and a single clone containing the onion sequence.

Table 4 shows the results of the cloning of the amplification product obtained from the freeze-dried packet soup (23 clones sequenced). The results obtained correspond to the composition indicated on the packet.

TABLE 4

| | | |
|---|---|---|
| Sequence obtained | Identification | Number |

| | | |
|---|---|---|
| ATCTTTATTTTTTGAAAAACAAGGGTTTA | leek | 9 |
| AAAAAGAGAATAAAAAAG | | |
| ATCCTGTTTTCTGAAAACAAACAAAGGTT | potato | 3 |
| CAGAAAAAAAG | | |
| ATCTTTCTTTTTTGAAAAACAAGGGTTTA | onion | 1 |
| AAAAAGAGAATAAAAAAG | | |

### 5) Comparative Example on a Degraded Substrate with the Primers g, h and c, d

The objective of this experiment was to compare the present invention with the approach published in 1991 (Taberlet P, Gielly L, Pautou G, Bouvet J (1991) Universal primers for amplification of three non-coding regions of chloroplast DNA. Plant Molecular Biology, 17, 1105-1109).

The genomic DNA was extracted from 100 mg of breadcrumbs using an extraction kit (Dneasy Plant Mini Kit, Qiagen) and according to the supplier's instructions. The final volume of the DNA extract was recovered in 100 μl.

The amplification was carried out using, firstly, the primers g and h and, secondly, the primers c and d (Taberlet P, Gielly L, Pautou G, Bouvet J (1991) Universal primers for amplification of three non-coding regions of chloroplast DNA. Plant Molecular Biology, 17, 1105-1109). The following amplification conditions were applied:

Final volume: 25 μl

MgCl₂: 2 mM

dNTP: 0.2 mM each

Primers: 1 μM each

Taq polymerase (Amplitaq Gold, Perkin Elmer): 1 unit

BSA: 0.2 μl per tube

Volume of DNA extract used: 2.5 μl ( 1/80 of the extract)

Initial denaturation of 10 nm at 95° C.

Number of cycles: 25

Denaturation: 30 s at 95° C., hybridization: 30 s at 55° C., extension: 60 s at 72° C.

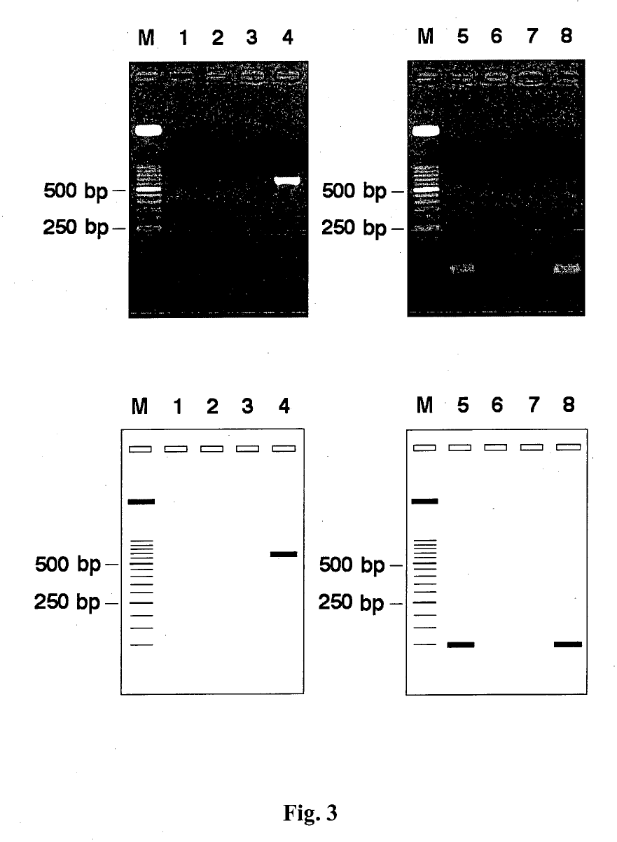

FIG. 3 shows the results obtained. No amplification product is apparent with the primers c and d, whereas an amplification product is obtained with the primers g and h.

1) Pair of oligonucleotides characterized in that the first oligonucleotide hybridizes to the SEQ ID No. 68 sequence and in that the second oligonucleotide hybridizes to the SEQ ID No. 69 sequence under stringency conditions which are sufficient for the selective amplification of a variable region of the intron of the trnL chloroplast gene of tobacco, whose sequence is represented at SEQ ID No. 3.

2) Pair of oligonucleotides characterized in that the first oligonucleotide hybridizes to the SEQ ID No. 68 sequence and in that the second oligonucleotide hybridizes to the SEQ ID No. 69 sequence under stringency conditions which are sufficient for the selective amplification of a variable region of the intron of the trnL chloroplast gene of plants whose sequence is represented at SEQ ID Nos. 24-67.

3) Pair of oligonucleotides according to claim 1, wherein hybridization occurs at 55° C. in an amplification buffer comprising 2 mM MgCl2.

4) Pair of oligonucleotides according to claim 1, wherein the first oligonucleotide is chosen from the group comprising SEQ ID Nos. 1, 4-15 and in that the second oligonucleotide is chosen from the group comprising SEQ ID Nos. 2, 16-23.

5) Oligonucleotide wherein its sequence is chosen from the group comprising SEQ ID No. 1, SEQ ID No. 2, SEQ ID Nos. 4 15, SEQ ID Nos. 16 23.

6) Polynucleotide wherein its sequence is chosen from the group comprising SEQ ID Nos. 24-67.

7) Pair of oligonucleotides according to claim 1, wherein the pair is immobilized on a solid support.

8) Method for amplifying a variable region of chloroplast DNA of plants, comprising:
a) providing a sample including plant genomic DNA;
b) amplifying a variable region of chloroplast DNA with a pair of oligonucleotides according to claim 1.

9) Method for amplifying a variable region of chloroplast DNA of plants according to claim 8, wherein, at step b), the variable region of amplified chloroplast DNA is a polynucleotide whose sequence is chosen from the group comprising SEQ ID Nos. 24-67.

10) Method for detecting a plant species in a sample, comprising:
a) providing a sample suspected of containing a plant species;
b) carrying out an amplification reaction with a pair of oligonucleotides according to claim 1; and
c) detecting whether an amplification product proving the presence of a plant species in the sample is obtained.

11) Method for detecting a plant species in a sample according to claim 10, wherein, at step c), the amplification product is a polynucleotide whose sequence is chosen from the group comprising SEQ ID Nos. 24 67.

12) Method for identifying a plant species in a sample, comprising:
a) providing a sample suspected of containing a plant species;
b) carrying out an amplification reaction is with a pair of oligonucleotides according to claim 1; and
c) analyzing the amplification product thus obtained to identify the plant species contained in the sample.

13) Method for identifying a plant species in a sample according to claim 12, in which, at step c), the amplification product is a polynucleotide whose sequence is chosen from the group comprising SEQ ID Nos. 24 67.

14) Method for identifying a plant species in a sample according to claim 12, in which, at step c), the sequence of the amplification product is determined for identifying the plant species contained in the sample.

15) Method for identifying a plant species in a sample according to claim 12, in which, at step c), the amplification product is hybridized with at least one reference plant sequence for identifying the plant species contained in the sample.

16) Method for identifying a plant species in a sample according to claim 15, in which the reference sequence is chosen from the group comprising SEQ ID Nos. 3 and 24-67.

17) Method for identifying a plant species in a sample according to claim 12, in which, at step c), the amplification product is analyzed by electro-phoresis for identifying the plant species contained in the sample.

18) Use of the variable region of the intron of the trnL chloroplast gene of plants corresponding to positions 49425 to 49466 of the chloroplast DNA of tobacco for detecting and identifying plant species.

19) Use according to claim 18 in which the variable region of the intron of the trnL chloroplast gene of plants is a polynucleotide whose sequence is chosen from the group comprising SEQ ID Nos. 24 67.


SEQUENCE LISTING (69 sequences)

SEQ ID NO: 1 - DNA, 17 bp, Nicotiana tabacum
gggcaatcctgagccaa

SEQ ID NO: 2 - DNA, 22 bp, Nicotiana tabacum
ccattgagtctctgcacctatc

SEQ ID NO: 3 - DNA, 40 bp, Nicotiana tabacum
atcctgttttccgaaaacaaacaaaggttcagaaaaaaag

SEQ ID NO: 4 - DNA, 17 bp, Prunus persica
gggcgatcctgagccaa

SEQ ID NO: 5 - DNA, 17 bp, Urtica dioica
gggcaatcctgaaccaa

SEQ ID NO: 6 - DNA, 17 bp, Artificial sequence
  Variants of the primer g
aggcaatcctgagccaa

SEQ ID NO: 7 - DNA, 17 bp, Artificial sequence
  Variants of the primer g
cggcaatcctgagccaa

SEQ ID NO: 8 - DNA, 17 bp, Artificial sequence
  Variants of the primer g
gggtaatcctgagccaa

SEQ ID NO: 9 - DNA, 17 bp, Artificial sequence
  Variants of the primer g
gggcgatcctgagccaa

SEQ ID NO: 10 - DNA, 17 bp, Artificial sequence
  Variants of the primer g
gggctatcctgagccaa

SEQ ID NO: 11 - DNA, 17 bp, Artificial sequence
  Variants of the primer g
gggccatcctgagccaa

SEQ ID NO: 12 - DNA, 17 bp, Artificial sequence
  Variants of the primer g
gggcaattctgagccaa

SEQ ID NO: 13 - DNA, 17 bp, Artificial sequence
  Variants of the primer g
gggcaatcctgggccaa

SEQ ID NO: 14 - DNA, 17 bp, Artificial sequence
  Variants of the primer g
gggcaatcctgacccaa

SEQ ID NO: 15 - DNA, 17 bp, Artificial sequence
  Variants of the primer g
gggcaatcctgatccaa

SEQ ID NO: 16 - DNA, 22 bp, Beta vulgaris
cctttgagtctctgcacctatc

SEQ ID NO: 17 - DNA, 22 bp, Phaseolus vulgaris
ccatagagtctctgcacctatc

SEQ ID NO: 18 - DNA, 22 bp, Cynara cardunculus
ccatcgagtctctgcacctatc

SEQ ID NO: 19 - DNA, 22 bp, Urtica dioica
ccgttgagtctctgcacctatc

SEQ ID NO: 20 - DNA, 22 bp, Artificial sequence
  Variants of the primer h
ccattgagtctctgtacctatc

SEQ ID NO: 21 - DNA, 22 bp, Artificial sequence
  Variants of the primer h
ccattgagtctcggcacctatc

SEQ ID NO: 22 - DNA, 22 bp, Artificial sequence
  Variants of the primer h
ccgtcgagtctctgcacctatc

SEQ ID NO: 23 - DNA, 22 bp, Artificial sequence
  Variants of the primer h
ccattgagtctctgcacctgtc

SEQ ID NO: 24 - DNA, 65 bp, Theobroma cacao
atcctattattttattattttacgaaactaaacaaaggttcagcaagcgagaataataaaaaaag

SEQ ID NO: 25 - DNA, 56 bp, Beta vulgaris
ctccttttttcaaaagaaaaaaaataaggattccgaaaacaagaataaaaaaaaag

SEQ ID NO: 26 - DNA, 55 bp, Castanea sativa
atcctattttacgaaaacaaataagggttcagaagaaagcgagaataaaaaaaag

SEQ ID NO: 27 - DNA, 55 bp, Cannabis sativa
atccggttttctgaaaacaaacaaggattcagaaagcaataataaaaaagaatag

SEQ ID NO: 28 - DNA, 54 bp, Cicer arietinum
atcctgctttcggaaaacaaacaaaaaaagttcagaaagttaaaatcaaaaaag

SEQ ID NO: 29 - DNA, 53 bp, Saccharum officinarum
atccccttttttgaaaaaacaagtggttctcaaactagaacccaaaggaaaag

SEQ ID NO: 30 - DNA, 53 bp, Asparagus officinalis
atctttatgtttagaaaaacaagggttttaatttaaaaactagaagaaaaagg

SEQ ID NO: 31 - DNA, 52 bp, Triticum aestivum
atccgtgttttgagaaaacaaggggttctcgaactagaatacaaaggaaaag

SEQ ID NO: 32 - DNA, 52 bp, Oryza sativa
atccatgttttgagaaaacaagcggttctcgaactagaacccaaaggaaaag

SEQ ID NO: 33 - DNA, 52 bp, Panicum miliaceum
atcccttttttgaaaaaacaagtggttctcaaactagaacccaaaggaaaag

SEQ ID NO: 34 - DNA, 52 bp, Ribes aureum
atcctgttttacaaacaaaacacaagagttcacaaagagagaataaaaaaag

SEQ ID NO: 35 - DNA, 52 bp, Fragaria vesca
atcccgttttatgaaaacaaacaagggtttcagaaagcgagaataaataaag

SEQ ID NO: 36 - DNA, 52 bp, Citrus X paradisi
atcctcttctcttttccaagaacaaacaggggttcagaaagcgaaaaagggg

SEQ ID NO: 37 - DNA, 52 bp, Triphasia trifolia; identical to SEQ ID NO: 36

SEQ ID NO: 38 - DNA, 51 bp, Vitis vinifera
atcctgttttccgaaaacaaccaagggttcagaaaacgataataaaaaaag

SEQ ID NO: 39 - DNA, 51 bp, Prunus persica
atcctgttttattaaaacaaacaagggtttcataaaccgagaataaaaaag

SEQ ID NO: 40 - DNA, 51 bp, Actinidia chinensis
atcctttttttcgaaaacaaacaaagattcagaaagcgaaaataaaacaag

SEQ ID NO: 41 - DNA, 51 bp, Zea mays
atcccttttttgaaaaacaagtggttctcaaactagaacccaaaggaaaag

SEQ ID NO: 42 - DNA, 51 bp, Pisum sativum
atccttctttctgaaaacaaataaaagttcagaaagtgaaaatcaaaaaag

SEQ ID NO: 43 - DNA, 50 bp, Phaseolus vulgaris
atcccgttttctgaaaaaaagaaaaattcagaaagtgataataaaaaagg

SEQ ID NO: 44 - DNA, 50 bp, Sorghum halepense
atccacttttttcaaaaaagtggttctcaaactagaacccaaaggaaaag

SEQ ID NO: 45 - DNA, 50 bp, Cynara cardunculus
atcacgttttccgaaactaaacaaaggttcagaaagcgaaaatcaaaaag

SEQ ID NO: 46 - DNA, 50 bp, Arctium lappa
atcacgttttccgaaaacaaacaaaggttcagaaagcgaaaataaaaaag

SEQ ID NO: 47 - DNA, 50 bp, Lactuca sativa
atcacgttttccgaaaacaaacaacggttcagaaagcgaaaatcaaaaag

SEQ ID NO: 48 - DNA, 50 bp, Helianthus annuus; identical to SEQ ID NO: 46

SEQ ID NO: 49 - DNA, 50 bp, Ficus carica
atccggttttctgaaaacaaacaagggttcagaaggcgataataaaaaag

SEQ ID NO: 50 - DNA, 49 bp, Humulus lupulus
atccggttttctgaaaacaaacaaggattcagaaagcaataataaaggg

SEQ ID NO: 51 - DNA, 48 bp, Avena sativa
atccgtgttttgagaggggggttctcgaactagaatacaaaggaaaag

SEQ ID NO: 52 - DNA, 48 bp, Nasturtium officcinale
atccttgtttacgcaaacaaaccggagtttagaaagcgagaaaaaagg

SEQ ID NO: 53 - DNA, 48 bp, Armoracia rusticana
atccttgtttacgcgaacaaacctgagtttagaaagcgagataaaagg

SEQ ID NO: 54 - DNA, 47 bp, Hordeum vulgare
atccgtgttttgagaagggattctcgaactagaatacaaaggaaaag

SEQ ID NO: 55 - DNA, 47 bp, Anthriscus cerefolium
atcctattttttccaaaaacaaacaaaggcccagaaggtgaaaaaag

SEQ ID NO: 56 - DNA, 47 bp, Allium cepa
atctttcttttttgaaaaacaagggtttaaaaaagagaataaaaaag

SEQ ID NO: 57 - DNA, 47 bp, Allium porrum
atctttattttttgaaaaacaagggtttaaaaaagagaataaaaaag

SEQ ID NO: 58 - DNA, 45 bp, Carum petroselinum
atcctattttccaaaaacaaacaaaggcccagaaggtgaaaaaag

SEQ ID NO: 59 - DNA, 40 bp, Solanum tuberosum
atcctgttttctgaaaacaaacaaaggttcagaaaaaaag

SEQ ID NO: 60 - DNA, 40 bp, Solanum lycopersicum
atcctgttttctgaaaacaaaccaaggttcagaaaaaaag

SEQ ID NO: 61 - DNA, 40 bp, Solanum melongena
atcctgttttctcaaaacaaacaaaggttcagaaaaaaag

SEQ ID NO: 62 - DNA, 39 bp, Raphanus sativus
atcctgagttacgcgaacaaaccagagtttagaaagcgg

SEQ ID NO: 63 - DNA, 39 bp, Brassica oleracea capitata
atcctgggttacgcgaacaaaacagagtttagaaagcgg

SEQ ID NO: 64 - DNA, 39 bp, Brassica nigra
atcctgggttacgcgaacaaaccagagtttagaaagcgg

SEQ ID NO: 65 - DNA, 39 bp, Olea europaea
atcctgttttcccaaaacaaaggttcagaaagaaaaaag

SEQ ID NO: 66 - DNA, 34 bp, Urtica dioica
atctggtgttataaaacaaagcgataaaaaaaag

SEQ ID NO: 67 - DNA, 31 bp, Rumex acetosa
ctcctcctttccaaaaggaagaataaaaaag

SEQ ID NO: 68 - DNA, 17 bp, Artificial sequence
  Sequence of the region complementary to the primer g
ttggctcaggattgccc

SEQ ID NO: 69 - DNA, 22 bp, Artificial sequence
  Sequence of the region complementary to the primer h
gataggtgcagagactcaatgg